(19) United States
(12) Patent Application Publication
(10) Pub. No.: US 2023/0347077 A1
(43) Pub. Date: Nov. 2, 2023

(54) CAPSULE INHALER

(71) Applicant: PULINNO SP. Z O. O., Bydgoszcz (PL)

(72) Inventors: Pawel ROSZCZYK; Tomasz SOSNOWSKI; Arkadiusz MOSKAL; Emil KLUCZ; Mscislaw SRUTEK; Slawomir BUJNOWSKI; Mateusz WIRWICKI; Waldemar WISNIEWSKI; Krzysztof KLUCZ

(21) Appl. No.: 18/042,807

(22) PCT Filed: Aug. 23, 2021
(86) PCT No.: PCT/IB2021/057718

§ 371 Date: Feb. 24, 2023

(30) Foreign Application Priority Data
Aug. 24, 2020 (PL) P.435071

(51) Int. Cl. A61M 15/00

(52) U.S. Cl. CPC A61M 15/0041; A61M 15/0026; A61M 2202/064; A61M 2205/583; A61M 2205/8206

(57) ABSTRACT

The invention provides a capsule inhaler for administering a single dose of dry powder from a capsule, and comprising a body and a mouthpiece with a sieve and a mouthpiece base, and a cover having an opening, coupled with the body. Additionally, the inhaler comprises a capsule receiving element and press buttons with a capsule piercing mechanism. In the body of the inhaler an electronic board is arranged with a microprocessor, said board comprising a magnetic field sensor and a light indicator, arranged in the proximity of the press button that comprises a magnet. The magnet is positioned at the piercing element, and in the proximity of the magnet a magnetic field sensor is arranged, and the electronic board of the inhaler is coupled with the mouthpiece base via a coupling element.

The invention provides a smart capsule inhaler of a variable resistance parameter, ensuring pulmonary deposition suitable for specific drug formulation in a dry powder form enclosed in a cellulose or gelatine capsule.

There are known devices for ensuring proper pulmonary deposition, both in a form of caps or accessories for inhalers, as well as smart inhalers as such having a structure that ensures proper administering of a full drug dose.

Document GB 2542910 A discloses a device and a method intended for ensuring proper intake of a drug with the use of a capsule inhaler that uses a dedicated band for its user. The band comprises a source of power, an inhaler proximity sensor, a controller, a memory, a time measurement unit and a communication unit. The band, by means of a detecting sensor periodically checks the proximity of the inhaler and this is enabled by a marker provided on the inhaler, and when the inhaler is detected it is activated to collect and record data provided from the inhaler and then transmit them to a home memory. One of the most important aspects of the invention is ensuring low power consumption by the inhaler and the band.

Devices are known from document U.S. Pat. No. 10,155,094 B2, which are capable to monitor the use of the inhaler, having a form of caps applied on the inhaler and enabling detection of the inhaler being used and transmitting information collected by the device during the use of the inhaler to the user. The device disclosed in U.S. Pat. No. 10,155,094 B2 may comprise a pressure sensor for detecting inhalation, a position sensor for detecting position and orientation of the inhaler, and a sensor or a switch to activate the monitoring device upon detection of pressure on the inhaler, in order to administer a drug dose or upon opening of the inhaler housing, as well as a memory for recording data obtained from the sensors during the drug intake operation. The device disclosed in document U.S. Pat. No. 10,155,094 B2 may be used solely for inhalers with a container for a liquid and due to its construction it does not provide a possibility to control piercing of the capsule with a drug, and it is not adaptable for capsule inhalers which limits its application. The aim of the solution of document U.S. Pat. No. 10,155,094 B2 is to provide a monitoring device releasable from the inhaler, to be used for numerous liquid inhalers, but just with regard to this functionality, the manner of assembling the monitoring device and absence of a module for possible connection to elements responsible for piercing of the capsule, the device is not applicable for capsule inhalers.

The specification of the invention P.422716 discloses an inhaler for a single dose of dry powder, of a construction enabling intake of a drug in a form of a capsule and ensuring pulmonary deposition dedicated to the respective drug formulation. The inhaler disclosed in document P.422716 comprises a mouthpiece, a capsule receiving chamber, and capsule piercing elements, as well as a rotational chamber, air inlet channels and a drug dispersing element. The inhaler ensures proper drug intake by ensuring suitable de-composition of the active substance particles, by means of its constructive solution, wherein the solution has adjustable constructive parameters and this enables adjusting it to specific drugs.

Devices known from the prior art offer methods to ensure that a dug has been administered to the user of an inhaler and that proper intake is effected, but they relate to other inhalers than the capsule-based ones. Apart from that, for complete monitoring they require additional devices such as bands, or they relate to caps and not to inhalers as such, and they do not verify correctness of piercing of the capsule with the drug. The solutions of the prior art do not provide automatic transmittal of complex instructions to the user and they require additional memory. There is a lack of solutions to ensure comprehensive monitoring of drug intake, dedicated for capsule inhalers, including inter alia verification of correctness of inhalation process and transmitting suitable feedback to the user in real time.

The capsule inhaler for administering a single dry powder dose from a capsule, comprising a body, a mouthpiece with a sieve and a mouthpiece base as well as a cover with an opening, coupled with the body, and a capsule receiving element and at least one press button with a capsule piercing mechanism, comprising at least one spring and at least one piercing element, is characterized in that in the body an electronic board with a microprocessor is arranged, comprising at least one magnetic field sensor and at least one light indicator, positioned in proximity of at least one press button comprising at least one piercing element and at least one magnet. The magnet is positioned by the piercing element and in proximity of the magnet the magnetic field sensor is positioned and the electronic board is coupled with the mouthpiece base via a coupling element.

Preferably, the magnetic field sensor is a Hall-effect device.

Preferably, the light indicator is a diode.

Preferably, the electronic board comprises a switch being a base opening sensor that, via a coupling element, is coupled with the mouthpiece base.

Preferably, the electronic board is arranged in a frame accommodated within the body.

Preferably, the coupling element is s pusher.

Preferably, the capsule receiving element comprises a pressure drop chamber and a capsule chamber, and in the lower part of the capsule receiving element, in the pressure drop chamber adjoining the capsule chamber a pressure sensor is arranged and positioned on the electronic board.

Preferably, on the electronic board, coupled with a microprocessor, an inhaler position sensor is arranged.

Preferably, the position sensor is an accelerometer.

Preferably, on the electronic board, coupled with a microprocessor, an antenna is arranged.

Preferably, the antenna is a Bluetooth antenna.

Preferably, on the electronic board, coupled with a microprocessor, a connector is arranged.

Preferably, the connector is a USB-C-type connector.

Preferably, on the electronic board, coupled with a microprocessor, an on/off switch is arranged.

Preferably, on the electronic board, coupled with a microprocessor, a resetting element is arranged.

Preferably, the resetting element is a resetting switch.

Preferably, on the electronic board, coupled with a microprocessor, a real tie clock is arranged.

Preferably, on the electronic board, coupled with a microprocessor, an audio indicator is arranged.

Preferably, in the frame a battery is arranged.

Preferably, the body is releasably coupled with the frame by means of at least one coupling element.

Preferably, the coupling element is a screw or a bolt.

Preferably, on the body and the cover an enclosing housing is provided covering the mouthpiece and the base.

Preferably, the sieve comprises a mesh with rectangular openings of a width comprised between 0.94 and 1 cm and a height comprised between 0.97 and 1.03 cm and a spacing between the openings between 0.47 and 0.53 cm.

Preferably, the sieve comprises a mesh with rectangular openings of a width comprised between 0.68 and 0.72 cm and a height comprised between 1.07 and 1.13 cm and a spacing between the openings between 1.37 and 1.43 cm.

Preferably, the sieve comprises a mesh with rectangular openings of a width comprised between 1.08 and 1.14 cm and a height comprised between 1.07 and 1.13 cm and a spacing between the openings between 0.90 and 0.96 cm.

Preferably, on the base, a protrusion is arranged for raising the base.

Preferably, the mouthpiece base is secured hingedly on the cover.

Inhaler according to the invention generates aerosol from a solid form of a drug comprising an active substance in a form embedded in a lactose carrier, under the flow of air caused during the user (patient) breath-in. This process requires overcoming internal resistance of the inhaler and aerodynamic resistance. Effectiveness of inhalation depends on forming of a breath-in flow ensuring proper disaggregation of the drug that determines generation of a micromolecular fraction. In turn, proper construction of the inhaler determines the rate and nature of pulmonary deposition to provide a high therapeutic effectiveness of the inhaled drugs. The inhaler is a one-dose device that requires a capsule with the drug to be inserted to the inhaler chamber and pierced by a needle mechanism.

The principal functionalities of the inhaler according to the invention include:
transmitting information concerning opening, closing of the inhaler;
measurement of pressure drop caused by the air flow in the inhaler;
possibility to determine the inhaler position in a XYZ coordinate system;
possibility to monitor motion of the press buttons that pierce the capsule with the drug;
possibility to establish a Bluetooth connection with a mobile phone;
possibility to record data from several measurements in the inhaler memory;
light and audio communication with the user;
dedicated application to enable collecting data from the device, with a function of training of the user for proper use of the inhaler;
application to indicate a high probability of intake of the whole therapeutic dose of a specific drug.

The object of the invention is shown in the drawing in which:

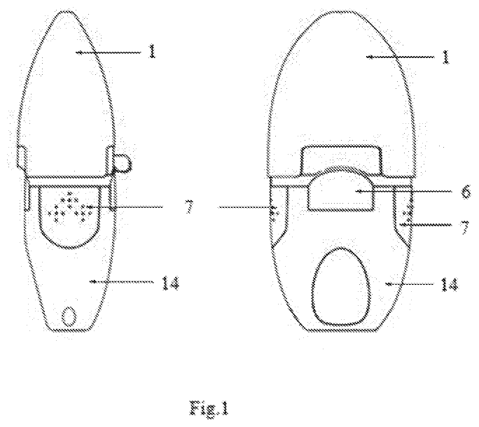

FIG. 1 shows an inhaler according to the invention, in a closed configuration with a cover, in a front and side views of the inhaler;

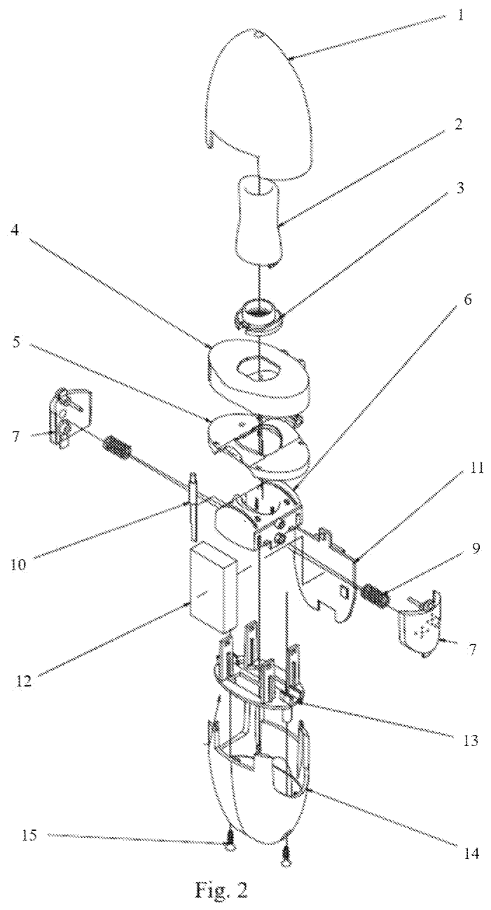

FIG. 2 shows an exploded view of an inhaler according to the invention;

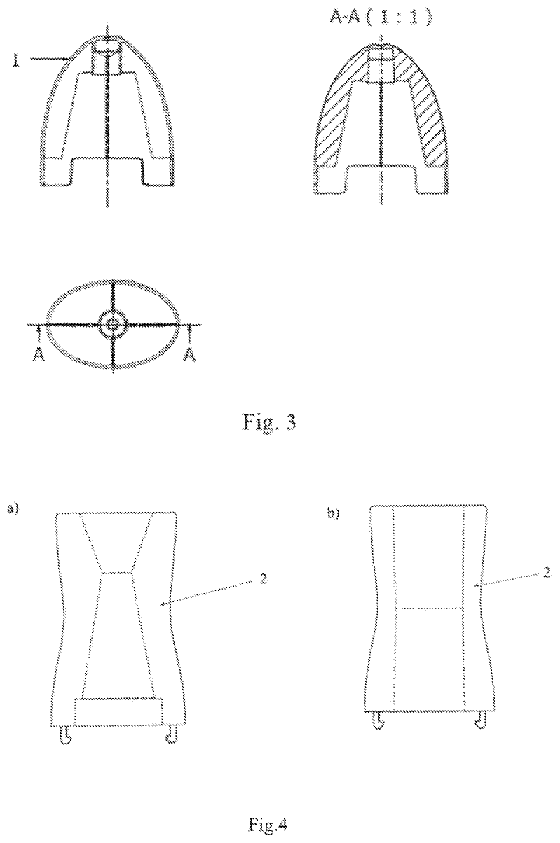

FIG. 3 shows a housing of an inhaler according to the invention, in cross-sectional views;

FIG. 4a shows a mouthpiece of an inhaler according to the invention with a dashed line indicating a channel within the mouthpiece and with visible catches;

FIG. 4b shows a mouthpiece of an inhaler according to the invention with a dashed line indicating a channel, other than the one in FIG. 4b, inside the mouthpiece and with visible catches;

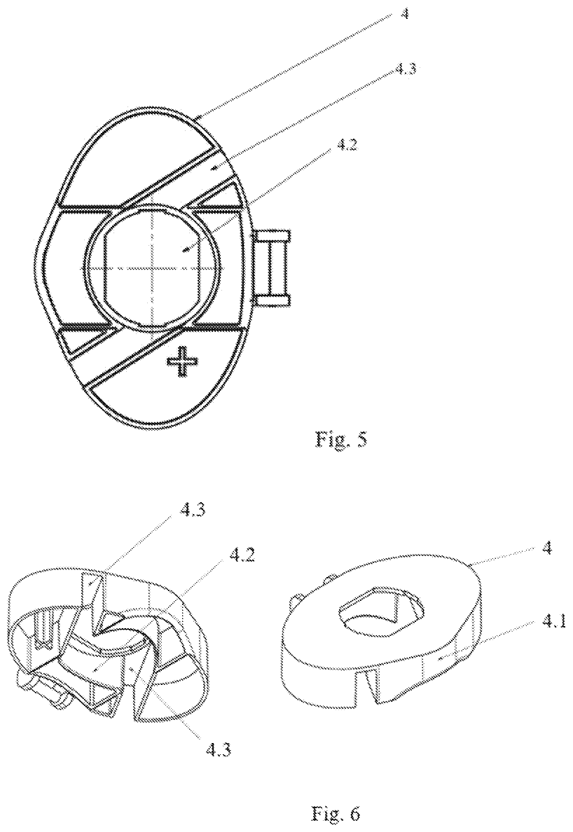

FIG. 5 shows a base of a mouthpiece of an inhaler according to the invention, in a bottom view with a visible inlet channel and a rotatable chamber;

FIG. 6 shows a base of a mouthpiece of an inhaler according to the invention, in an isometric bottom view and an isometric top view, respectively;

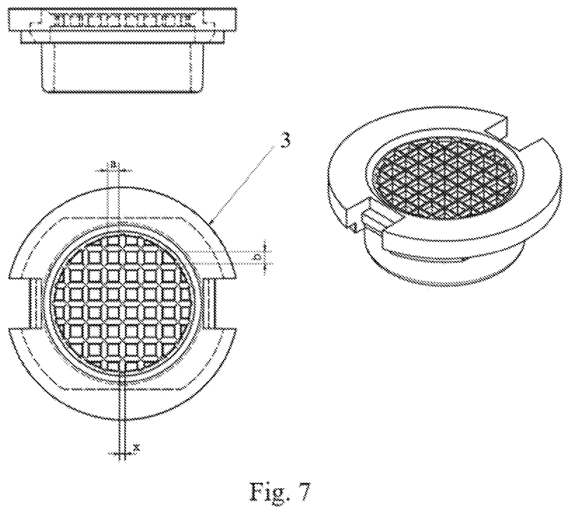

FIGS. 7-9 show sieves in side, top and isometric views;

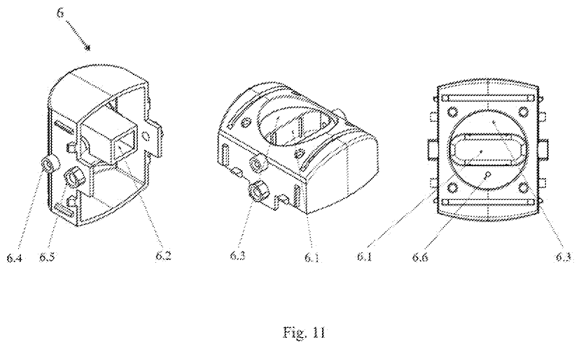

FIG. 10 shows a cover in an isometric top view;

FIG. 11 shows a capsule receiving element, in an isometric view presenting the bottom of the element, isometric view presenting the top of the element and in a top view of the element, respectively;

FIG. 12 shows a piercing mechanism of a press button of an inhaler according to the invention;

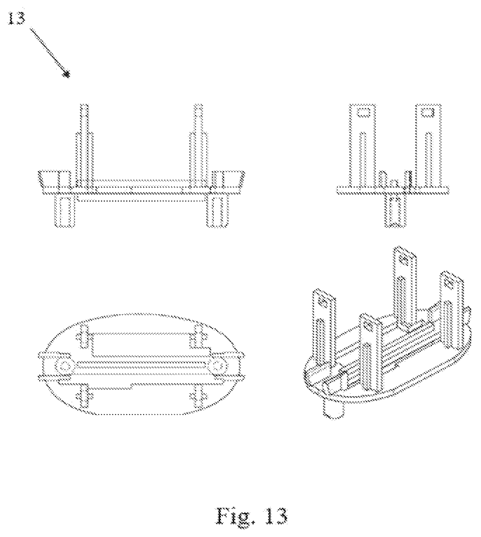

FIG. 13 shows a frame of an inhaler according to the invention;

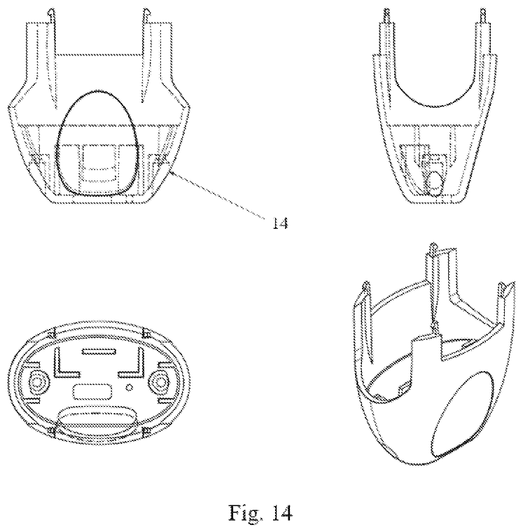

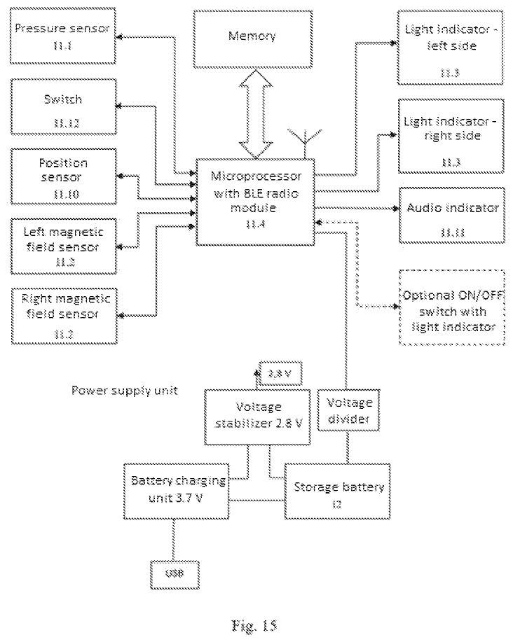

FIG. 14 shows a body of an inhaler according to the invention;

FIG. 15 shows a power supply diagram of an inhaler according to the invention;

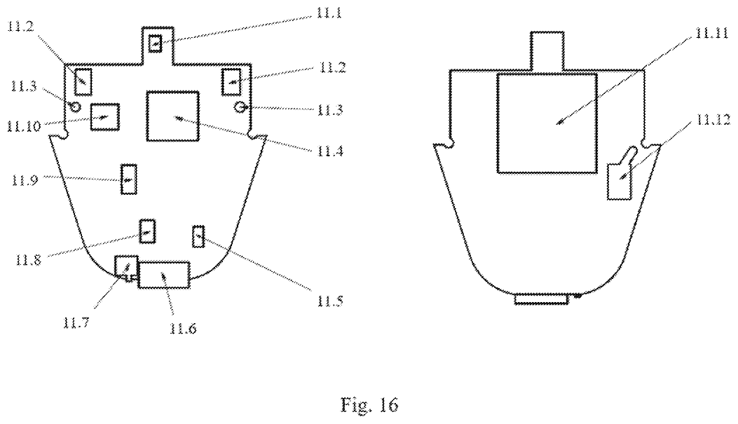

FIG. 16 shows an electronic board of an inhaler according to the invention.

In the embodiment shown in FIGS. 1 and 2, a capsule inhaler for administering a single dose of dry powder from a capsule comprises a body 14 coupled via catches with a cover 5 which is hingedly coupled to a base 4 of a mouthpiece 2. In an opening in the upper part of the base 4, a sieve 3 is mounted and the mouthpiece 2 that is closable from above by a housing 1. In the central “inner” part of the base 4 there is a rotatable chamber 4.2, to which, from two opposite sides, inlet channels 4.3 open, and through the channels air is drawn. On the base 4, a protrusion 4.1, for example, is provided to raise the base 4.

The cover 5 covers a capsule receiving element 6 which for example may be made of transparent plastics to enable monitoring the inside of the element to determine whether a capsule got stuck, or whether it is properly inserted, or whether it has been pierced. The capsule receiving element 6 in the outer-central-upper part has a volume 6.3 along with the capsule chamber 6.1 into which the user locates a capsule and in this chamber 6.1 the capsule is pierced. Within the volume 6.3 the capsule gets drawn by the air flow from the chamber 6.1 onto the capsule and swirls within the rotatably chamber 4.2 provided in the base 4 of the mouthpiece 2, to release therapeutic substance. However, within the inner bottom part of the capsule receiving element 6, one of the long sides of the capsule chamber 6.1 adjoins a pressure drop chamber 6.2, communicating via an opening 6.6 with the volume 6.3 and into which a part of the electronic board 11 with a pressure sensor 11.1 is inserted from beneath.

The pressure sensor 11.1 enables the user to measure drug dosing intensity and time. Pressure measurement in a function of time makes it possible to determine duration time and force with which the user makes a breath-in during drug intake.

The capsule receiving element 6 on each of its two opposite walls has two openings—an opening 6.4 for a piercing element and below an opening 6.5 for a mandrel. Into the opening 6.4 for a piercing element, a piercing element 7.1, for example a needle, enters. The piercing element 7.1 is a part of the piercing mechanism and it is secured to a press button 7 over the mandrel 7.2. Into the opening 6.5 for a mandrel, a mandrel 7.2 enters, said mandrel being also secured on the press button 7. On the mandrel 7.2 a spring 9 is provided and the mandrel 7.2 as such stabilizes the motion of the press button 7 so that the press button 7 when pressed does not deviate from its longitudinal axis and the piercing element 7.1 always “enters” the same orientation via the opening 6.4 into the capsule chamber 6.1 and provides for reproducibility of piercing of the capsule, and thus reproducible effectiveness of drug release. Additionally, on the press button 7 below the mandrel 7.2 a magnet 8 is provided. The press buttons 7 are positioned at the height of the capsule receiving element 6.

The inhaler has two press buttons 7 coupled with the body 14 and cover 5 via upper and bottom catches. In the body 14 a frame 13 is located, releasably coupled with the body 14 by means of two connecting elements 15, for example screws or bolts. The frame 13, shown in FIG. 13, enables precise positioning of the pressure sensor 11.1 relative to a specially designed pressure drop chamber 6.2. The frame 13 provides stabilization and smooth movement of the press buttons 7 and joins all the constructive elements. In an embodiment, in the frame 13 an electronic board 11 with a microprocessor 11.4 is positioned as well as a battery (or a storage cell) 12. In another embodiment the electronic board 11 is accommodated within a suitably profiled body 14.

The electronic board 11 has at its sides two magnetic field sensors 11.2, for example a Hall-effect device, and light indicators 11.3, for example LEDs, positioned each in the proximity of the press buttons 7. Additionally, the magnetic field sensors 11.2 are positioned in the proximity of magnets so that they can react to the motion of the magnets 8 that move over the magnetic field sensors 11.2, and this makes it possible to record the velocity and depth at which each of the piercing elements 7.1 is pressed.

In addition, the electronic board 11 also comprises a switch 11.12, being a base opening sensor 4, coupled with the base 4 of the mouthpiece 2 via a coupling element 10, for example a pusher, that passes through an opening in the upper surface of the cover 5, which surface adjoins the base 4 of the mouthpiece 2. Activation of the inhaler is effected upon opening of the base 4 of the mouthpiece 2. The status of the pusher is changed mechanically by opening the base 4 of the mouthpiece 2—detection of opening of the base 4 of the mouthpiece is recorded just thanks to the switch 11.12 and indicates initiation of the inhalation process.

In another embodiment, on the electronic board 11, coupled with the microprocessor 11.4, a position sensor 11.10 is arranged, such as for example 3-axial accelerometer that enables verification whether inhalation has been executed properly by verification whether the inhaler during inhalation was kept in a proper position, i.e. whether it had an inclination angle within the range 10°-20°+/−2° to the horizon, for example 15°.

In a further embodiment, on the electronic board 11, coupled with the microprocessor 11.4, an antenna 11.5 is arranged, for example a Bluetooth antenna, to enable wireless communication of the inhaler with a user device, such as for example a smartphone. The smartphone may have a suitable application installed therein and the principal functionalities of the application include:—
educational module—operation of the device, breath-in force and duration time, inclination angle of the device;
inhalation history along with daily/weekly/monthly statistics;
counting down for the next inhalation;
possibility of setting personalized reminders;
statistics module;
map of pollen along with personalized warning alerts;
personalized list of taken drugs along with reminders;
weather widget—temperature, air quality.

In one embodiment, on the electronic board 11, coupled with the microprocessor 11.4, a connector 11.6 is also positioned, for example a USB-C-type connector, to enable charging of the device powered by means of a battery 12 that gets discharged during operation.

In another embodiment, on the electronic board 11, coupled with a microprocessor, 11.4 an on/off switch is arranged that enables turning on and/or turning off the inhaler manually if for example power consumptions should be limited when the inhaler is unused.

In a further embodiment, in case of problems in operation of the inhaler, the settings of the device may be reset, and this is possible due to implementation, on the electronic board 11, of a resetting element 11.7, for example a resetting switch, connected to a microprocessor 11.4.

In another embodiment, on the electronic board 11, coupled with a microprocessor 11.4, a real time clock 11.8 is arranged, to enable during autonomous operation of the device without pairing to a mobile application, recording the precise time when measurement is taken.

In another embodiment, inhaler, on the electronic board 11, coupled with a microprocessor 11.4, has an audio indicator 11.11, that along with the light indicator 11.3 at each step of the inhalation process, when errors occur, transmits a corresponding alert to the user.

In a further embodiment, the inhaler comprises a replaceable sieve 3 to enable personalization of the inhaler to drugs to be administered to a specific user. For example, the sieve 3 shown in FIG. 7 may have a mesh with rectangular openings of a width a of 0.97 mm+/−0.03 mm and a height b of 1.00 mm+/−0.03 mm and a spacing X between the openings of 0.5 mm+/−0.03 mm.

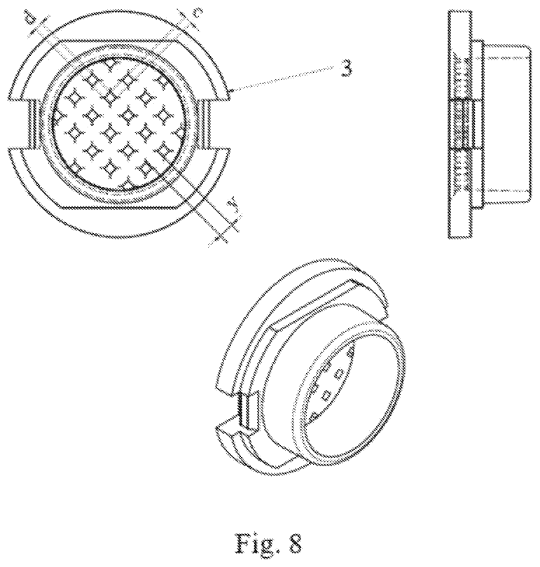

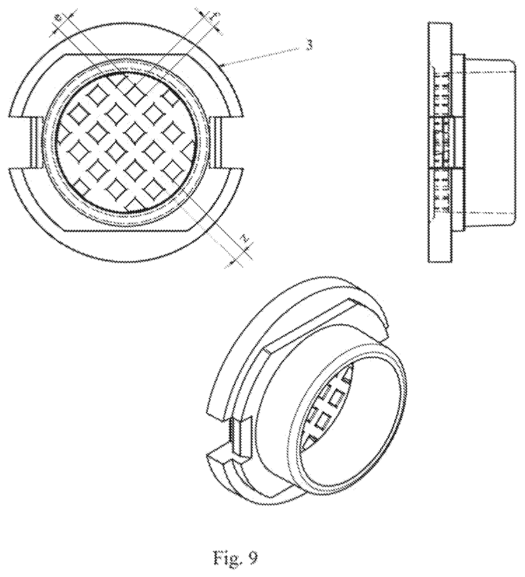

In another embodiment, the sieve 3 shown in FIG. 8 comprises a mesh with rectangular openings of a width c of 0.7 mm+/−0.02 mm and a height d of 1.1 mm+/−0.03 mm and a spacing Y between the openings of 1.4 mm+/−0.03 mm.

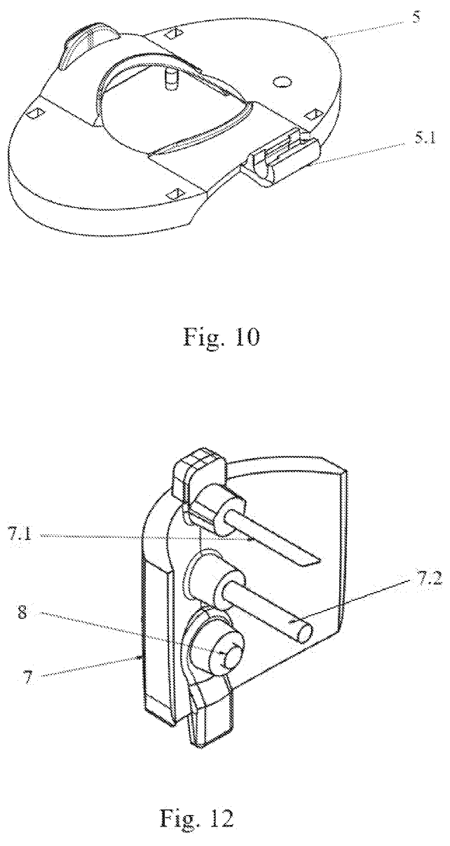

In a further embodiment, the sieve 3 shown in FIG. 9 comprises a mesh with rectangular openings of a width e of 1.11 mm+/−0.03 mm and a height f of 1.1 mm+/−0.03 mm and a spacing Z between the openings of 0.93 mm+/−0.03 mm.

In another embodiment, the inhaler has a replaceable mouthpiece 2 with a decreased clearance of the inner diameter of the mouthpiece 2 as shown in FIG. 4a.

In a further embodiment, the inhaler has a replaceable mouthpiece 2 with a constant value of the clearance of the inner diameter of the mouthpiece 2, as shown in FIG. 4b.

In a further embodiment, the electronic board 11 of the inhaler shown in FIGS. 15 and 16 comprises a microprocessor with a BLE radio module, for example CC2650F128RGZR, connected to the following:—
pressure sensor 11.1, for example BMP 280;
switch 11.2, for example SKRTLAE010;
position sensor 11.10, for example MPU6050;
left and right magnetic field sensor 11.2, for example 634-SI7210-B-00-IV and 634-SI7210-B-03-IV;
left and right light indicator 11.3, for example KPFA-3010RGBC-11;
audio indicator 11.11, for example SMT-1640-S-2-R;
optional on/off switch, for example with RGB LED signalling;
memory 11.9 such as EEPROM, for example AT24CM02, to enable the inhaler to record several complete operational cycles.

Additionally, in an embodiment, the microprocessor 11.4 has a power supply unit shown in FIG. 15, comprising: a voltage stabilizer 2.8 V (for example TC1015-2.8), LiPo storage battery 3.7 V minimum 380 mAh, voltage divider, storage battery charging unit 3.7 V (for example LTC4054) coupled with a connector 11.6 (for example a USB-C socket).

User who wishes to perform inhalation with the use of an inhaler according to the invention should prepare the device for use, first of all by checking whether the device is charged. Blinking blue light shows that the device is ready to be used and that it is not linked with a smartphone. Before the first use, the user decides whether he/she wishes to use a specially designed application to monitor regularity of the inhalations performed. The application has also an educational module aimed at training the user in proper performing of the therapeutic process (inhalation).

If the user wishes to operate the inhaler with the use of the application, he/she has to download it first to his/her smartphone. The user installs the application in his/her smartphone. Before activation of the application the user activates the Bluetooth module in the phone and performs pairing with the device. Upon installation of the application the user configures it according to the user's guide.

The user with inhaler thus prepared, with the application activated and with a blister of capsules with drug, initiates inhalation process. The user holds the inhaler in one hand and with the other hand he/she grasps the housing 1 of the device or with his/her thumb levers the protrusion 4.1. (FIG. 6) positioned on the base 4 of the mouthpiece 2 to open the device. The module mouthpiece unit that comprises the mouthpiece 2 (FIG. 4), sieve 3 (FIG. 7-9) and base 4 of the mouthpiece 2 (FIGS. 5 and 6) and the housing 1 (FIG. 3) opens upwards and pivots rearwards from the device. This opening of the device causes that pressure on the coupling element 10 (FIG. 2) is released. The movement resulting from releasing of the coupling element 10 also releases pressure on the switch 11.12 (FIG. 16b). The switch 11.12 is a part of electronics that controls the inhaler. The switch 11.12 is positioned on the electronic board 11 (FIGS. 2 and 16), positioned inside the body 14 of the inhaler (FIG. 14). The electronic board 11 may be for example a PCB made in four-layer technology, with components at both sides thereof (FIG. 16).

Release by the coupling element 10 of the pressure on the switch 11.12 causes generating of a signal read by the software that controls the inhaler operation as “device open”. The signal transmitted by the electric element, when generating the information “device open”, causes release of a light signal by diodes 11.3 positioned on the same electronic board 11. The diodes 11.3, at the moment when the inhaler is activated, start blinking green. At the same time, due to the use of the Bluetooth module being also the antenna 11.5, the microprocessor 11.4 positioned on the electronic board 11 sends a signal to the user's smartphone. In the application screen a message “Insert capsule and close the device” appears. At this moment the user removes a capsule from a blister and positions it in a special capsule chamber 6.1 arranged in a capsule receiving element 6 (FIG. 11). When this operation is completed, the user closes the inhaler. The module mouthpiece system comprising the mouthpiece 2 (FIG. 4), sieve 3 (FIG. 7-9) and the base 4 of the mouthpiece 2 (FIGS. 5 and 6) and the housing 1 (FIG. 3) is closed downward to pivot the front of the device. If after the device is open the user does not perform any operation, after about 3 minutes the device will return to a standby mode.

After the capsule is in place and the device is closed, the user grasps the devices so that he/she encloses the inhaler with his/her hand. The application screen sends a message: “When keeping the device vertically press and release side press buttons. Green light will indicate that the capsule has been pierced properly”. The back side of the inhaler defines a hinge 5.1, positioned on the cover 5 (FIG. 10). The back of the inhaler is enclosed by the user's palm and thus the user may press simultaneously, using his/her index finger and thumb, the press buttons 7 (FIG. 12), positioned at both sides of the device. Before the press buttons 7 are pressed, the user may raise the device at the level of his/her eyes and state, looking through the capsule receiving element 6 (FIG. 11), whether in fact the capsule is positioned within the capsule chamber 6.1. If it is so, the user maintains the inhaler vertically and presses concurrently the press buttons 7. Needles 7.1 arranged on the press buttons 7, due to the slide movement of the press buttons 7 enabled by the use of springs 9 (FIG. 2), pierce the capsule. In the press buttons 7, magnets 8 are mounted (FIG. 12). The electronic board 11 has at its sides two Hall-effect sensors, “Hall-effect devices” 11.2 that react to the movement of the magnet 8. The signal transmitted by the Hall-effect devices 11.2 generates a numeric value of the signal. Trespassing of a threshold value causes the microprocessor installed or mobile application to approve the process and this is indicated to the user by activation of steady green light. If for some reason a suitable signal value is not reached, the diodes 11.3 will turn purple and the application will show a message “Purple light indicates that the capsule has been pierced improperly. Replace it an initiate inhalation again.”.

If the user has pierced the capsule properly, the press buttons 7 are released to return to the initial position due to the use of the spring 9. The signal generated by the Hall-effect devices 11.2 caused by the movement of the magnets 8 has returned to the initial status and then the user receives the following information from the application: “Before inhalation remove the cap and make a shallow breath-in outside the inhaler area. Next put the inhaler mouthpiece into your mouth.”. The device is ready for further process and thus it sends steady green light. Upon removal of the housing 1 (FIG. 2), the user clicks “Next” in the application. The user receives a message “Find the proper angle for the inhaler. Remember to keep upright posture”.

The user makes a movement with the inhaler to put it into the vertical position. Before the user makes an inhalation breath-in, he/she has to set the device at the proper angle. Setting of the proper angle is possible thanks to the accelerometer 11.10 positioned on the electronic board 11. A change in the angle of the device causes the software that controls the device or the mobile application to monitor its position within the XYZ coordinate. During searching of the proper angle, the diodes 11.3 are off. But when the proper angle is set they start sending steady green light. Additionally, reaching of the proper angle is signalled by the inhaler by means of a single audio signal. In the application a message appears “When keeping the proper posture take a deep breath-in” and two arrow heads appear at the sides of the screen. When the device reaches the proper angle, the arrow heads in the screen merge into one line and change their colour into green. The application, in addition to the light signalling also generates a single audio signal. This is possible thanks to the use of the audio indicator 11.11 arranged on the electronic board 11. When the user hears the audio signal, he/she makes a breath-in, inserts the inhaler mouthpiece 2 into his/her mouth and clenches it. The user makes a strong and possibly quick breath-in. The device, due to the use of the pressure sensor 11.1 arranged on the electronic board 11, may monitor pressure drop in the inhaler.

Pressure drop dP, correspondingly to the type of the inhaler used, effects the flow volume of the air Q caused by the user breathing-in. During the breath-in the pressure drop causes the air to flow. The air is drawn into the device by two inlet channels 4.3. The air reaches the rotatable chamber 4.2 arranged in the base 4 of the mouthpiece 2. The air generates a swirl and at the same time a negative pressure that draws the pierced capsule from the capsule chamber 6.1. The capsule makes a rotary motion and releases the drug through the openings resulting from piercing. The drug is composed of two kinds of particles: fine particles of the therapeutic substance and coarse particles of the carrier. These two particles are joined during the drug manufacturing process under mechanic forces and not chemical bonds. As a result of drawing the agglomerate from the capsule, as well as circulation of air, the agglomerate breaks into two fractions, a fine one and a coarse one. The capability of the user to draw the proper amount of air determines effectiveness of de-agglomeration of the drug structure. As a result, an aerosol is produced i.e. a mixture of air with therapeutic substance and carrier particles suspended therein. During breathing-in by the user, the aerosol reaches upper respiratory tract. Effective de-aggregation increases chance for the fine particle fraction comprising the therapeutic substance to reach the lower portion of the respiratory tract and become “absorbed” into the bronchioles. Obtaining of the proper air flow is a necessary condition for high probability of providing a proper therapeutic dose. With the use of a formula to determine relations between the pressure drop and the air flow, optimum ranges of pressure drop to be obtained are determined that are required to enable a considerable probability of providing proper therapeutic dose. The pressure sensor 11.1 positioned on the electronic board 11, during assembly of the device, is inserted into a specially designed pressure drop chamber 6.2, that adjoins the capsule chamber 6.1, and the pressure drop chamber 6.2 is communicated via an opening 6.6 with a volume 6.3 that is positioned along with the capsule chamber 6.1 in the upper part of the capsule receiving element 6.

The user breaths in. The device sends steady green light until a pressure change is stated. The diodes 11.3 blink green. At this time the user should hold his/her breath for 5 seconds. The diodes 11.3 blink till a minimum time of 5 seconds elapses. The application counts down 5 seconds. When the minimum of 5 seconds is reached, the device sends steady green light. The application shows a message: “Have you held your breath for 5 seconds?”.

The user breathes the air out. When the user confirms or denies holding his/her breath, the application generates a message “Have you removed the tablet?”. The user opens the inhaler similarly as at the beginning. Release of the coupling element 10 causes that a signal is generated to the device and to the application. The user cleans the inhaler and closes it. Upon confirming, the user receives an inhalation process report. When the user clicks “Next” he/she receives a message to confirm complete inhalation and to show the time for the next inhalation. When the button “End” is pressed, two audio signals are generated, and the inhaler buttons send steady green light. After 3 seconds the device passes into a standby phase.

As used herein, all terms “upper”, “lower” or the like throughout this text are provided as an example and do not limit the positioning of the inhaler elements or configuration thereof.

The embodiments shown herein are solely non-limiting indications related to the invention and cannot in any way limit the scope of protection defined by the patent claims. It should be understood that each technical solutions used in the inhaler according to the invention may be implemented by means of equivalent technologies without exceeding the scope of protection.

1. A capsule inhaler for administering a single dose of dry powder from a capsule, said inhaler comprising:
a body,
a mouthpiece having a sieve, a base, and a cover comprising an opening, the mouthpiece being coupled with the body,
a capsule receiver,
at least one press button with a capsule piercing mechanism comprising at least one spring, at least one piercing element, and at least one magnet, and at least one magnet
an electronic board is arranged in the body and having a microprocessor, said board comprising at least one magnetic field sensor and at least one light indicator, positioned in proximity to the at least one press button, wherein the magnet is positioned in proximity to the piercing element and in proximity to the magnetic field sensor, and
a first coupler coupling the electronic board with the base (4) of the mouthpiece.

2. The inhaler according to claim 1, wherein the magnetic field sensor is a Hall-effect device and the light indicator is a diode.

3. (canceled)

4. The inhaler according to claim 1, wherein the electronic board is arranged in a frame accommodated within the body, and comprises a switch being a base opening sensor that via the first coupler is coupled with the base of the mouthpiece.

5.-6. (canceled)

7. The inhaler according to claim 1, wherein the capsule receiver comprises a pressure drop chamber and a capsule chamber, and at the lower part of the capsule receiver, in the pressure drop chamber adjoining the capsule chamber, a pressure sensor is arranged on the electronic board.

8. The inhaler according to claim 1, further comprising an inhaler position sensor and an antenna, the inhaler position sensor and the antenna being arranged on the electronic board and coupled with a microprocessor.

9-11. (canceled)

12. The inhaler according to claim 1, further comprising a connector arranged on the electronic board, coupled with a microprocessor.

13. (canceled)

14. The inhaler according to claim 1, further comprising an on/off switch arranged on the electronic board, coupled with a microprocessor.

15. The inhaler according to claim 1, further comprising a resetting element arranged on the electronic board, coupled with a microprocessor.

16. The inhaler according to claim 15, wherein the resetting element is a resetting switch.

17. The inhaler according to claim 1, further comprising a real time clock arranged on the electronic board, coupled with a microprocessor.

18. The inhaler according to claim 1, further comprising an audio indicator arranged on the electronic board, coupled with a microprocessor.

19. The inhaler according to claim 1, further comprising a battery arranged in a frame.

20. The inhaler according to claim 19, wherein the body is releasably coupled with the frame by at least one second coupler.

21. The inhaler according to claim 20, wherein the at least one second coupler is a screw or a bolt.

22. The inhaler according to claim 1, further comprising a housing positioned on the body and the cover that encloses and covers the mouthpiece and the base.

23. The inhaler according to claim 1, wherein the sieve comprises a mesh with rectangular openings of a width (a) comprised between 0.94 and 1 cm and a height (b) comprised between 0.97 and 1.03 cm and a spacing (X) between the openings of from 0.47 to 0.53 cm.

24. The inhaler according to claim 1, wherein the sieve comprises a mesh with rectangular openings of a width (c) comprised between 0.68 and 0.72 cm and a height (d) comprised between 1.07 and 1.13 cm and a spacing (Y) between the openings of from 1.37 to 1.43 cm.

25. The inhaler according to claim 1, wherein the sieve comprises a mesh with rectangular openings of a width (e) comprised between 1.08 and 1.14 cm and a height (f) comprised between 1.07 and 1.13 cm and a spacing (Z) between the openings of from 0.90 to 0.96 cm.

26. The inhaler according to claim 1, further comprising a protrusion arranged on the base for raising the base.

27. The inhaler according to claim 1, wherein the base of the mouthpiece is coupled hingedly on the cover.
